(19) United States
(12) Patent Application Publication
(10) Pub. No.: US 2020/0013315 A1
(43) Pub. Date: Jan. 9, 2020

(54) SYSTEM AND METHOD FOR VALIDATING AND TRAINING SURGICAL INTERVENTIONS IN HUMAN AND VETERINARY MEDICINE

(71) Applicant: PHACON GMBH, Leipzig (DE)

(72) Inventors: Hendrik MÖCKEL, Leipzig (DE); Robert HAASE, Leipzig (DE)

(73) Assignee: PHACON GMBH, Leipzig (DE)

(21) Appl. No.: 16/482,847

(22) PCT Filed: Feb. 9, 2018
(86) PCT No.: PCT/EP2018/053274

§ 371 Date: Aug. 1, 2019

(30) Foreign Application Priority Data
Feb. 10, 2017 (DE) 10 2017 202 164.0
Feb. 10, 2017 (DE) 10 2017 202 165.9

(51) Int. Cl. G09B 23/34; G16H 20/40

(52) U.S. Cl. CPC G09B 23/34; G16H 20/40

(57) ABSTRACT

The invention relates to a system for validating and training surgical interventions in human and veterinary medicine, which includes a training model anatomically modelled after a human or animal body. The system comprises an anatomical reproduction of a body part of the human or animal body, which has a recess and an anatomical reproduction of an interchangeable practice region, which is designed to be insertable into the recess of the body part. The interchangeable practice region has a front side and a rear side, wherein the rear side is connected at least in part in an interlocking manner to the recess of the anatomical reproduction of the body part. The system also comprises an optoelectronic detection means, which is situated in the recess of the anatomical reproduction of the body part such that it is designed for detecting the surgical interventions and for monitoring the positioning of surgical instruments and/or implants on the rear side of the interchangeable practice region. The invention further relates to an anatomical reproduction of an interchangeable practice region, which may be inserted in a holding device, as well as a method for validating and training surgical interventions in human and veterinary medicine using a training model anatomically modelled after a human or animal body, wherein optoelectronic detection means mounted in the interchangeable practice region of the training model sends a feedback signal to the control, measuring and evaluation unit and thus monitors and evaluates the training process in real time.

The invention relates to a system and a method for validating and training surgical interventions in human and veterinary medicine, which includes a training model anatomically modelled after a human or animal body.

In the process, parameters are established that provide information about the learning success, the quality and thus a success monitoring of the practiced intervention. The practiced interventions comprise surgical and minimally invasive surgical techniques such as, for example, setting implants, which are trained under realistic conditions on the training model.

The training model includes an anatomical reproduction of a body part, an anatomical reproduction of an interchangeable practice region and an optoelectronic detection means, wherein the optoelectronic detection means is situated in the recess of the anatomical reproduction of the body part in such a way that this detection means is designed to detect the surgical interventions and to monitor the positioning of surgical instruments and/or implants on the rear side of the interchangeable practice region. The training model also comprises a memory medium, which is connected in a detachable manner to the interchangeable practice region.

The surgical intervention to be practiced is supported by a data record. An authentic injury to risk structures (for example, of vessels or nerves) is automatically shown.

The object of the present method is validating and training surgical interventions in a training model anatomically modelled after a human or animal body with the aid of the system according to the invention.

Various validation and training methods are known, in which training models are used for training surgeons and for advance testing of complex surgical and minimally invasive surgical interventions prior to the actual operation.

US 2001/0019818 A1 describes an anatomical model for training in heart operations using endoscopic methods. The model includes anatomically realistically simulated bones and organs as well as layers of skin. The organs may be designed transparent for better presentation. The possibility of providing illumination inside the model is also described. However, the model allows no automated control and feedback to the user with respect to the interventions performed.

US 2014/0057236 A1 describes a combined system of an anatomical model for training surgical interventions and a virtual representation of the surgical field. In this case, a virtual representation of the surgical field is generated via a display situated between the operating surgeon and the model, while the operating surgeon operates simultaneously using the underlying model. As a result, an overall perception is produced, which is intended to produce a preferably realistic feeling for the surgeon. The system also provides feedback to the user on the proper positioning of boreholes in bones. This is intended to enable the user to learn the exact positioning of the holes. A disadvantage of the system, however, is the limited perception created as a result of the limited virtual representation.

WO 2012/044753 A2 discloses a portable device for training in laparoscopic interventions. The system is suitable for practicing endoscopic interventions. The model may also include LED illumination below the surface for illuminating simulated body cavities.

WO 2013/165529 A2 discloses a system and a method for analysing surgical techniques, wherein the course of the surgical intervention is recorded and evaluated via optoelectronic detection means. For this purpose, some layers of the model contain dyes that are detected by the system.

WO 2015/138982 A1 describes a model for training in laparoscopic interventions. In this case, the operating surgeon focuses on the structuring and design of the muscle layers located under the simulated skin layer. In this way, a wrong access point is identified. To check the correct puncture site, the model may be turned over and inspected from the rear side. However, it is necessary to terminate the intervention in this case. Moreover, this model does not allow for direct monitoring of the surgical techniques.

Finally, DE 11 2006 003 722 B4 discloses a simulation system for surgical interventions in human and veterinary medicine.

It would therefore be highly desirable to provide a system, which captures the ongoing processes within the training model and which informs the user in real time of the training success and positioning of the surgical instruments and/or implants.

DE 20 2012 011 452 U1 discloses a neurosurgical training system for the planning and implementation of a craniotomy in brain tumors. The training system consists, among other things, of a phantom designed as a skull, a control, measuring and evaluation unit as well as a unit for detecting and monitoring the tracking of the instruments used in training. The control, measuring and evaluation unit in this case includes a memory unit for storing individual patient data records.

DE 10242953 A1 describes a device for training in simulated navigation-assisted surgical interventions. In this case, surgical instruments are controlled by a navigation system. There is also a patient data record obtained by imaging methods such as, for example, tomographic scans, as well as three reference points, which are not located in one plane and which are assigned to the body of the patient in a fixed position. The navigation system is equipped with a model of the body of the patient, which includes three additional reference points detectable by the navigation system. In this case, the patient data record is assigned to the model in such a way that the respective reference points coincide.

The document DE 10143712 A1 discloses a method, a computer system and a computer program product for data evaluation and improved patient care. The data analysis is intended to be used as an Internet-based, patient-specific prognosis system, in particular, for cancer diseases. The data set contains clinical pathological data and information on aftercare. A reference database is also used for comparison.

DE 10 2008 014 709 A1 discloses an image-guided expert surgical system, which provides access as well as storage and exchange of pieces of medical information between imaging devices during the performance of surgical procedures. In this case, the captured data are compared with reference data relating, for example, to a medical record, in order to improve the procedures.

EP 1348393 A1 describes a method for computer-aided medical navigation. In this method, the position of the patient and the position of treatment devices are detected by a position detection unit. Body structure data are assigned to these positional data. The body structure data was obtained on the basis of a generic model, which has been adapted by being linked to patient characteristic recorded data.

DE 10 2013 109 057 A1 discloses a method for planning and preparing an operative intervention in the human or animal body. At least one implant is intended to be used in this method. In the process, multi-dimensional representations of the body, which are based on reference databases, are shown in each case via an image assistance device.

Finally, DE 112006003722 B4 discloses a simulation system for surgical interventions in human and veterinary medicine, in which structures that mark permitted areas and risk areas are introduced into an anatomical model. In conjunction with the detection of the surgical instruments, it is possible as a result to identify the approach, contact and injury of forbidden zones. This is controlled in turn by an electronic control, measuring and evaluation unit, with which the chances of success of a practiced surgical intervention are simultaneously evaluated.

A disadvantage of these documents is that the patient data are stored in external databases separate from the actual training model, which require time-consuming access until the desired data can be used. Moreover, these databases contain all the parameters of all recorded patients, as a result of which the user is required to first choose in a time-consuming and in partly convoluted manner which record he/she wants to use for each practice situation. Further disadvantages are the time-consuming updating of the entire databases which, among other things, are also dependent on the existing Internet connection, as well as in case of disruptions, the potential loss of all data records in the database. This results in an ineffective use of the aforementioned systems.

It would therefore be highly desirable to provide a training system which allows the user a user-friendly use by the user being able to determine the desired training to be performed using components that may be assembled in variable ways.

The object of the invention is therefore to provide a system which overcomes the disadvantages of the prior art.

A training system is to be provided which is suitable for validating and training in variously designed surgical interventions in human and veterinary medicine, and which is individually adapted and designed for such purpose. The individual design of the training system is intended to ensure an effective, specialized and focused training.

According to the invention, the object is achieved by a system, by an anatomical reproduction of an interchangeable practice region and by a method according to the independent claims. Advantageous embodiments are specified in the dependent claims

A first aspect of the invention relates to a system for validating and training surgical interventions in human and veterinary medicine, which includes a training model anatomically modelled after a human or animal body. The system comprises an anatomical reproduction of a body part of the human or animal body having a recess and an anatomical reproduction of an interchangeable practice region, which is designed to be inserted into the recess of the anatomical reproduction of the body part, and which has a front side accessible from the outside, and a rear side, which is connected in an interlocking manner to the recess of the anatomical reproduction of the body part. The system further comprises an optoelectronic detection means. The optoelectronic detection means is situated in the recess of the anatomical reproduction of the body part in such a way, that it is designed to detect the surgical interventions and to monitor the positioning of surgical instruments and/or implants on the rear side of the interchangeable training region by means of the control, measuring and evaluation unit.

In the process, the optoelectronic detection means is used to detect the processes occurring within the training model and to inform the user in real time about the training success and the positioning of the surgical instruments and/or of the implants. The surgical field is thus monitored in an automated manner.

The system also comprises an electronic control, measuring and evaluation unit as well as means for signal transmission.

The system according to the invention is used to evaluate newly developed surgical systems and surgical instruments, and/or implants for training surgeons and for testing complicated surgical interventions prior to the actual operation to increase the chances of a successful surgical intervention in various surgical disciplines.

The course and the result of the surgical intervention are recorded and presented objectively and in real time.

The training model is used by the user, hereinafter also referred to as operating surgeon, surgeon or doctor, for validating and training surgical interventions in human and veterinary medicine.

In one embodiment, the system is designed as a simulation system. The system includes a training model. In this training model, surgical and minimally invasive surgical techniques are simulated and tested by reproducing real anatomical and functional conditions in the patient during a surgical or minimally invasive surgical intervention.

The training model represents a precise and anatomically accurate reproduction of a human or animal body.

According to the invention, the training model comprises the anatomical reproduction of a body part, an anatomical reproduction of an interchangeable practice region and an optoelectronic detection means.

According to the invention, the system comprises an anatomical reproduction of a body part of the human or animal body. The term “anatomical reproduction of the body part” within the context of the invention refers to various body parts, all of which may be anatomically reproduced. A body part is understood to be a segment of the body recognizable morphologically as a functional unit. This includes, for example, leg, arm, pelvis, head or spine.

In one embodiment, the anatomical reproduction of the body part of a patient has various configurations. The correspondingly reproduced human or animal body part is anatomically precise and realistically reproduced. The type of design of the body part is based on various factors such as, for example, the recreated age of the patient to be examined and/or of the corresponding anatomy. In one embodiment, each body part of a human or animal may be represented as an anatomical reproduction of a body part.

The size of the anatomical reproduction of a body part of a person in the context of the invention refers to any size of a body part that a person, male or female, may assume in the course of his or her life. In one embodiment, the size of the body part refers to the reproduced realistic anatomy of an average adult male or of an average adult female. In another embodiment of the invention, the size of the body part refers to a child, and corresponds in this case to the size of the body part of each reproduced realistic anatomy of a child. The size of the body part of an animal within the context of the invention refers to any body part size, which an animal, whether male or female, may have in the course of its life. In one alternative embodiment of the invention, the size of the body part is smaller or larger than the reproduced realistic anatomy in a human or animal.

The type of design of the anatomical reproduction of a body part is further based on the course of the disease and/or on the stage of the disease of the patient to be examined.

In one preferred embodiment, the anatomical reproduction of the body part is designed as an anatomical reproduction of a skull, particularly preferably, as an anatomical reproduction of a human skull.

According to the invention, the anatomical reproduction of the body part has a recess. A recess within the context of the invention is understood to be a recessed space or an opening or a cavity in the anatomical reproduction of the body part. The recess may have different dimensions and is adapted to the size of the body part and to the interchangeable practice region, which may be inserted into the recess. The recess in the anatomical reproduction of the body part corresponds to a space into which at least part of the interchangeable practice region may be inserted.

In one embodiment, the anatomical reproduction of a body part is designed as a skull, wherein the recess is located in the area of the petrous bone region of the skull. In one alternative embodiment, the recess is located in the area of the nasal bone region of the skull.

In another alternative embodiment, the anatomical reproduction of a body part is designed as an arm, as a forearm in one specific embodiment, wherein the recess is located in the area of the arteries on the radial and ulnar sides.

In another alternative embodiment, the anatomical reproduction of a body part is designed as a back region, wherein the recess is located in the area of the spinal column. In this case, either the entire spine may be shown or only a section such as, for example, the cervical spine, thoracic spine or lumbar spine.

According to the invention, the system comprises an optoelectronic detection means. In one preferred embodiment, the optoelectronic detection means is situated in the recess of the anatomical reproduction of the body part in such a way that it is designed to detect the surgical interventions and to monitor the positioning of the surgical instruments and/or implants on the rear side of the interchangeable practice region by means of the electronic control, measuring and evaluation unit.

In one embodiment, the optoelectronic detection means is used to detect the processes occurring within the training model and to inform the user in real time about the training success and the positioning of the surgical instruments and/or the implants. The surgical field is thus monitored in an automated manner.

In one preferred embodiment, the optoelectronic detection means is connected by signal transmission means to the electronic control, measuring and evaluation unit.

In one embodiment, a feedback signal from the optoelectronic detection means is sent by signal transmission means to the electronic control, measuring and evaluation unit. In one embodiment, the signal transmission means are electrical cables.

In one preferred embodiment, the optoelectronic detection means is a recording device, very preferably a video digital camera. In one alternative embodiment, the optoelectronic detection means is a webcam. In one preferred embodiment, the optoelectronic detection means has a CMOS sensor. In one alternative preferred embodiment, the optoelectronic detection means has a CCD sensor, specifically, a two-dimensional CCD array sensor.

In one preferred embodiment, the feedback signal of the optoelectronic detection means mounted in the interchangeable practice region of the training model is transmitted to the electronic control, measuring and evaluation unit and evaluated and analysed by the computer program product, so that the training process is monitored and evaluated in real time. The duration of training and the number of injuries are also registered by the computer program product.

In one embodiment, the optoelectronic detection means is connected by signal transmission means to the electronic control, measuring and evaluation unit. In one embodiment, the signal transmission means are electrical cables.

In one embodiment, the optoelectronic detection means transmits, via the signal transmission means, a signal fed back with the optoelectronic detection means to the electronic control, measuring and evaluation unit. The transmitted signal contains data about the current training process. The data includes audio-visual specifications such as image resolution and derived aspect ratio, refresh rate, and color depth.

In one embodiment, the optoelectronic detection means transmits the recorded data during operation, i.e., during training, to the electronic control, measuring and evaluation unit. In another embodiment, the optoelectronic detection means transmits the recorded data before or after the training to the electronic control, measuring and evaluation unit.

The data with the interchangeable practice region are thus transmitted to the electronic control, measuring and evaluation unit when the interchangeable practice region is connected to the anatomical reproduction of the body part.

By situating the optoelectronic detection means in the recess of the anatomical reproduction of the body part, the surgical interventions and techniques taking place during the training are advantageously transmitted by the signal transmission means to the electronic control, measuring and evaluation unit. This ensures a monitoring of the positioning of the surgical instruments and/or implants on the rear side of the interchangeable practice region.

In one embodiment, the risk structures to be examined and/or the anatomical space surrounding the risk structures are designed to be transparent and thus advantageously facilitate good visualization for the optoelectronic detection means. In one preferred embodiment, at least portions of the interchangeable practice region are transparent. The risk structure is defined as those components in the interchangeable practice region of the training model that may be injured during the training but should not be injured when used on the living patient.

In one embodiment, plastics such as polyamides or silicones, for example, are used as the transparent material.

The optoelectronic detection means also advantageously ensures a monitoring of the success of the intervention on the training model, for example, by estimating how far an implant, such as, for example, a cochlear implant, may be inserted. The optoelectronic detection means advantageously monitors the vulnerable risk structures and thus injuries in the interchangeable practice region. The user is also informed by the optoelectronic detection means in real time about the training success. Training success includes, for example, the positioning of the surgical instruments and/or the implants, for example, how far a cochlear implant could be introduced into the interchangeable practice region.

The user is informed by the feedback signal of the optoelectronic detection means in real time about the training process, which is monitored and evaluated.

The training may be advantageously prematurely interrupted in the event of errors and thus save time, or the training may be continued later or restarted.

Errors occurring are understood to mean, for example, a wrong passage or positioning of the surgical instruments and/or the implants on the rear side of the interchangeable practice region caused by incorrect positioning of the surgical instruments and/or the implants on the front side of the interchangeable practice region, which is situated locally at different location on the rear side of the interchangeable practice area from that required and planned. The number of injuries of functionally important anatomical areas, which were caused by a wrong passage or positioning of the surgical instruments and/or the implants, are registered in the process.

This also includes a holder for the anatomically reproduced body part, so that it is stored stably and against slippage during training.

During the intervention, statements may advantageously be made about possible injured risk structures and thus injuries, and the success of the operation may thus be tracked. The success monitoring or the learning success offer the user certainty and routine during the surgical interventions. The course of the operation is advantageously also evaluated. For example, an assessment is made regarding the manner of positioning of the surgical instruments and/or the implants or how much of the tissue to be removed of the interchangeable practice region or transparently formed risk structures contained therein and the anatomical space surrounding the risk structures was removed.

According to the invention, the system comprises an anatomical reproduction of an interchangeable practice region.

The term “interchangeable practice region” within the context of the invention refers to a body region which is anatomically topographically and functionally assigned to the corresponding defined anatomical reproduction of a body part. The interchangeable practice region corresponds to the surgical field for the user of the training model in which he/she practices surgical interventions. In one embodiment, the practice region is interchangeable. The interchangeable practice region represents an anatomical reproduction of a body region. In one embodiment, each body region of a human or animal may be represented as an interchangeable practice region.

According to the invention, the interchangeable practice region may be inserted into the recess of the anatomical reproduction of the body part. In one embodiment, the interchangeable practice region is designed to fit into the recess of the anatomical reproduction of the body part in an interlocking manner.

According to the invention, an anatomical reproduction of an interchangeable practice region may be inserted into a holding device. An anatomical reproduction of an interchangeable practice region within the context of the invention has a front side, which is accessible from the outside, and a rear side, which is connected to the holding device in an interlocking manner. In one embodiment, the holding device is designed as a technical support. The anatomical reproduction of the interchangeable practice region may be introduced into any conceivable holding device in an interlocking manner.

In one preferred embodiment, the anatomical reproduction of an interchangeable practice region, designed to be inserted into the holding device, comprises an optoelectronic detection means. In another preferred embodiment, at least sections of the anatomical reproduction of an interchangeable practice region, designed to be inserted into the holding device, are partially transparent in design. In another preferred embodiment, the anatomical reproduction of an interchangeable practice region, designed to be inserted into the holding device, also comprises a memory medium. In another preferred embodiment, the anatomical reproduction of an exchangeable training region, designed to be inserted into the holding device, comprises signal transmission means.

In one preferred embodiment, the interchangeable practice region is detachably connected to the anatomical reproduction of the body part.

In one preferred embodiment, the interchangeable practice region is designed as a temporal bone, in particular as a precise anatomical reproduction of the petrous bone region, particularly preferably as a human petrous bone region, which may be inserted into a skull. In another embodiment, the interchangeable practice region is designed as a precise anatomical reproduction of the nasal bone region, which may be inserted into a skull. In one alternative embodiment, the interchangeable practice region is designed as a precise anatomical reproduction of an arm segment, in one specific embodiment, of a forearm segment, which may be inserted into the recess in the area of the arteries on the radial and ulnar side. In another alternative embodiment, the interchangeable practice region is designed as a precise anatomical reproduction of a vertebral column segment, which may be inserted into the recess of the back region. In this case, either the complete spine may be shown or only a section such as, for example, the cervical spine, thoracic spine or lumbar spine.

The type of design of the interchangeable practice region is based on various factors such as, for example, the recreated age of the patient and/or of the corresponding anatomy.

The size of the interchangeable practice region of a human within the context of the invention refers to any size of the interchangeable practice region that a human, male or female, may assume in the course of his or her life. In one embodiment, the size of the interchangeable practice region refers to the reproduced realistic anatomy of an average adult male or of an average adult female. In another embodiment of the invention, the size of the interchangeable practice region refers to the reproduced realistic anatomy of a child, and corresponds to the size of the anatomy of the respective child's age. The size of the interchangeable practice region of an animal within the context of the invention refers to any size of the interchangeable practice region that an animal, whether male or female, may have in the course of its life. In an alternative embodiment of the invention, the size of the interchangeable practice region is smaller or larger than the reproduced realistic anatomy in a human or animal.

Furthermore, the type of design of the interchangeable practice region is based on the course of the disease and/or on the stage of the disease of the patient to be examined.

According to the invention, the interchangeable practice region has a front side. In one embodiment, the front side is an area that faces outward and is exposed. Within the context of the invention, “outside” means the surrounding environment of the system from which the user has free access to the practice region. The front side is preferably accessible to the user from the outside, i.e., the surgical instruments and/or the implants are introduced through the front side into the interchangeable practice region. The front side of the interchangeable practice region thus represents the area of access for the user during training.

In one embodiment, the interchangeable practice region is opened by a surgical method using surgical instruments prior to the introduction of surgical instruments and/or implants. Thus, training in the opening of the interchangeable practice region advantageously occurs before, for example, the introduction of an implant. In one embodiment, the interchangeable practice region has an imitation skin, which has different and lifelike strengths and mechanical properties, so that an actual cutting through the imitation skin takes place by means of surgical instruments.

In an alternative embodiment, the front side of the interchangeable practice region is not in physical contact with the anatomical reproduction of the body part.

According to the invention, the interchangeable practice region also has a rear side.

In one embodiment, the rear side represents a surface facing inwards, i.e., for anatomically reproducing the body part.

In one embodiment, the rear side is at least partially in physical contact with the anatomical reproduction of the body part. Physical contact is understood according to the invention to mean an interlocking connection. In one embodiment, the rear side is at least partially in physical contact with the recess of the anatomical reproduction of the body part. In one preferred embodiment, the rear side is at least partially connected in an interlocking manner to the recess of the anatomical reproduction of the body part. In one embodiment, the rear side is in physical contact with the recess of the anatomical reproduction of the body part.

In one embodiment, the rear side of the interchangeable practice region is at least partially in physical contact with the anatomical reproduction of the body part. In one embodiment, the rear side is not accessible to the user from the outside.

In one embodiment, the anatomical reproduction of the body part and/or the interchangeable practice region may be produced using additive manufacturing methods based on three-dimensional patient data. For example, the anatomical reproduction of the body part and/or the interchangeable practice region may be produced using a rapid prototyping method. Thus, a patient-specific anatomy of the anatomical reproduction of the body part or of the interchangeable practice region may advantageously also be presented, as a result of which each training is individually designed and may be individually carried out.

In one preferred embodiment of the invention, the interchangeable practice region comprises a memory medium. In one embodiment, the rear side of the interchangeable practice region comprises a memory medium.

The memory medium is also referred to as data memory. In one embodiment, a semiconductor memory serves as a memory medium. In one preferred embodiment, the memory medium is a memory chip. In one embodiment, the memory medium is designed as a non-volatile data memory such as, for example, as a flash memory.

In one embodiment, data records are stored on the memory medium. In one preferred embodiment, patient-specific data are stored on the memory medium. In another embodiment, specific parameters for the design of the interchangeable practice region are stored on the memory medium.

A training model specifically adapted to appropriate anatomies and disease histories, and thus an effective and targeted use of the training model, is advantageously enabled as a result of the patent-specific data provided. The specific parameters for the design of the interchangeable practice region also provide indications about the body region to be examined.

In one embodiment, the patient-specific data stored on the memory medium comprise parameters relating to the anatomy of the patient, to the age of the patient, to previous findings, to pathogenesis, to the clinical picture, as well as to already existing evidence of imaging methods, such as CT or X-ray images, which are used as templates and serve as orientation, and which allow the training situation to appear as realistic as possible. In one embodiment, the patient-specific data correspond to the medical record of the respective patient to be examined. The operating surgeon may advantageously visualize the training upcoming and to be performed before the practice intervention. Thus, the intervention to be trained is more realistically designed.

In another embodiment, specific parameters for the design of the interchangeable practice region, and thus for the body region to be examined, are stored on the memory medium. Thus, the body region to be examined is advantageously not only an anatomical reproduction of the interchangeable practice region, but also encompasses all the specific parameters required (design and geometry of the interchangeable practice region) for carrying out the training.

In one preferred embodiment, the memory medium may be written or overwritten with additional patient-specific data. This is done by additionally writing and adding these data to the memory medium or by overwriting the original patient-specific data with these data. Additional patient-specific data are understood to mean either extended findings of the same patient or the new data of another patient having a different clinical picture and anatomy and age, which are written completely new or in addition to and stored on the memory medium. Thus, the user is always able to practice new interventions on the training model. Furthermore, the control electronics of the memory medium are always updatable, as a result of which errors such as tampering or malfunctions may be reduced.

In one embodiment, the memory medium is mechanically connected to the interchangeable practice region in a detachable manner. In one preferred embodiment, the memory medium is detachably situated in the interchangeable practice region. In another embodiment, the memory medium is detachably situated in the rear side of the interchangeable practice region. Thus, the memory medium may be advantageously removed from and reconnected to the interchangeable practice region for writing with additional or new patient-individual data, for writing with additional or new specific parameters for the design of the interchangeable practice region or for updating the control electronics.

Because of the detachable arrangement of the memory medium in the interchangeable practice region, it is possible to connect different memory media, each of which contains different patient-specific data and specific parameters for the design of the interchangeable practice region, to the interchangeable practice region and thus to train in different clinical pictures for the same designed anatomical interchangeable practice region.

In one embodiment, the memory medium is mechanically and electrically connected in a detachable manner to the anatomical reproduction of the body part.

In one preferred embodiment, the memory medium is connected by signal transmission means to the electronic control, measuring and evaluation unit.

According to the invention, the interchangeable practice region may be inserted into the recess of the anatomical reproduction of the body part. In one embodiment, the interchangeable practice region is designed to be interlocking in a detachable manner in the recess of the anatomical reproduction of the body part. In one embodiment, the interchangeable practice region is mechanically connected to the anatomical reproduction of the body part.

Leading from the anatomical reproduction of the body part are signal transmission means which, in turn, are connected to the electronic control, measuring and evaluation unit.

In one embodiment, the signal transmission means are electrical cables. In one alternative embodiment, the signal transmission means are a wireless connection, which is designed, for example, as Bluetooth or WLAN.

The anatomical reproduction of the body part and the memory medium of the interchangeable practice region form a detachable plug connection. This ensures an electrical connection as well as a data connection, specifically, as a hardware interface.

In one preferred embodiment, the anatomical reproduction of the body part is connected to the interchangeable practice region and the memory medium contained in the interchangeable practice region with the patient-specific data provided thereon, and the specific parameters for the design of the interchangeable practice region are recognized by the electronic control, measuring and evaluation unit. This is done by transmitting and assigning the patient-specific data and the specific parameters for the design of the interchangeable practice region to the electronic control, measuring and evaluation by signal transmission means. This ensures an exchange of data when connecting the memory medium in the interchangeable practice region to the anatomical reproduction of the body part to the electronic control, measuring and evaluation unit.

Thus, the memory medium advantageously circumvents a time-consuming access to a complete database as described, for example, in DE 20 2012 011 452 U1, which contains all patient-specific data and which must, for example, first download or transmit these data. A quick and independent access to the patient-specific data and to the specific parameters of the interchangeable practice region is advantageously ensured without, for example, having to update a complete database.

A quick access to the patient-specific data contained on the memory medium and specific parameters for the design of the interchangeable practice region is advantageously possible at any time. The system is not dependent on a central database to which there is access, for example, via an Internet connection and where it would thus be necessary to ensure a consistently reliable connection. Thus, the training model may also be used at locations having no Internet connection. In addition, the patient-specific data and the specific parameters for the design of the interchangeable practice region are advantageously unaffected, for example, in the case of a system crash, since they are not stored centrally in a database, but on the memory media.

By transmitting and displaying the patient-specific data when connecting the interchangeable practice region to the anatomical reproduction of the body part, it becomes immediately apparent which patient (age, anatomy, previous findings, pathogenesis, clinical picture, existing evidence of imaging procedures such as, for example, CT or X-ray images) is involved and what surgical interventions the user must perform. By simultaneously transmitting and displaying the specific parameters regarding the respective design of the interchangeable practice region to be examined when connecting the interchangeable practice region to the anatomical reproduction of the body part, it also becomes immediately apparent which anatomical reproduction of the body region is involved.

In one preferred embodiment, the system comprises surgical instruments. In one preferred embodiment, the surgical instruments provided comprise various tools from the surgical and minimally surgical fields, such as, for example, pointing device, drill, spherical plugger, surgical scissors, surgical forceps, suction device or medical endoscope.

The surgical instruments are advantageously positioned by the user exactly at the desired and medically required location.

In one embodiment, the selected and medically required surgical instruments are positioned on the front side of the interchangeable practice region and passed through the interchangeable practice region to the rear side of the interchangeable practice region. The passage and positioning of the surgical instruments on the rear side of the interchangeable practice region are visible as a puncture site in the imitation skin.

In one embodiment, the surgical instruments include a marking, which includes identifying features and parameters. The marking is advantageously variously designed, so that the surgical instruments are distinguishable from one another. The marking on the surgical instruments is detected by a detection means, such as a camera, and forwarded to a computer program product, which recognizes which surgical instruments are used in each training. In one embodiment, the detection means also monitors the surgical intervention on the front side of the interchangeable practice region.

In one embodiment, any commercially designed surgical instrument is suitable for training in surgical interventions with the system according to the present invention. In one embodiment, the surgical instruments are designed as attachments.

In one embodiment, the surgical instruments are connected by signal transmission means to the electronic control, measuring and evaluation unit. In one embodiment, the signal transmission means are electrical cables.

The surgical instruments are individually adapted to the training to be performed and may be advantageously exchanged depending on the type of training or in case of repairs.

In one preferred embodiment, the system comprises implants. In one embodiment, the implants comprise medical implants such as, for example, a cochlear implant.

The implants are advantageously positioned by the user exactly at the desired and medically required location.

In one embodiment, the selected and medically required implants are positioned on the front side of the interchangeable practice region and passed through the interchangeable practice region to the rear side of the interchangeable practice region. The passage and positioning of the implants on the rear side of the interchangeable practice region are visible as a puncture site in the skin imitation.

In one embodiment, the implants are connected by signal transmission means to the electronic control, measuring and evaluation unit. In one embodiment, the signal transmission means are electrical cables.

The implants are individually adapted to the training to be performed and may be advantageously exchanged depending on the type of training or in case of repairs.

In one preferred embodiment, the system comprises an electronic control, measuring and evaluation unit. The electronic control, measuring and evaluation unit ensures a virtual and/or realistic representation of the examination in the surgical field during training on the training model.

In one preferred embodiment, the system comprises signal transmission means. The electronic control, measuring and evaluation unit is connected to signal transmission means. This allows automated monitoring of the interchangeable practice region and of the surgical field during operation, i.e., during the training. In one embodiment, the signal transmission means are electrical cables.

A virtual representation is understood to mean a three-dimensional reproduction of the training process in the surgical field of the interchangeable practice region, which is generated by the computer program product. In this case, medical image material stored on the memory medium serves as the basis for the virtual representation, which is generated by a computer program product.

A realistic representation is understood to mean a visual reproduction of the training process in the surgical field of the interchangeable practice region, which is transmitted, for example, as a video image in real time by an optoelectronic detection means.

The virtual and/or realistic representation of the surgical field is transmitted to the electronic control, measuring and evaluation unit, while the user operates simultaneously using the underlying training model.

In one embodiment, the electronic control, measuring and evaluation unit is a computer.

In one embodiment, the electronic control, measuring and evaluation unit further comprises a computer program product, which provides for the virtual and/or realistic representation of the surgical field and training process.

In one embodiment, the surgical scenarios to be learned may be studied using procedural protocols. In the procedural protocols, parameters are defined which provide, as success monitoring, information on the quality of the intervention being performed and thus (in the case of several tests) on learning success. The parameters are, for example, the duration of the procedure, the economy of hand movement, the injury of functionally important anatomical areas.

In one embodiment, the computer program product further recognizes the surgical instruments and/or implants used during training on the basis of the marking applied to them and calibrates them accordingly for the respective training situation. Thus, the training and the training process are controlled and monitored on the training model.

In one embodiment, the surgical instruments are calibrated in order to determine the axial lengths and location of the tip of the surgical instruments. A direction calibration also takes place, with which to determine the orientation of the surgical instruments within the virtual and/or realistic representation of the surgical field.

In another embodiment, the interchangeable practice region used therein with the specific parameters for the design of the interchangeable practice region contained in its memory medium, as well as patient-specific data, is recognized by the computer program product when connecting the interchangeable practice region to the anatomical reproduction of the body part.

In another embodiment, a patient calibration may be carried out, in which the coordinate system generated in the virtual and/or realistic representation of the interchangeable practice region located in the recess of the anatomical reproduction of the body part is aligned with the coordinate system of the surgical instruments.

In one preferred embodiment, the feedback signal of the optoelectronic detection means mounted in the interchangeable practice region of the training model is transmitted to the electronic control, measuring and evaluation unit and evaluated and analysed by the computer program product, so that the training process is monitored and evaluated in real time. The duration of training and the number of injuries are also registered by the computer program product.

In one preferred embodiment, the system and the method according to the invention are used for validating and training surgical interventions in human and veterinary medicine. In one embodiment, the system is intended for practice by apprentices, specialists, trainees or system testers. In another embodiment, the system is intended for companies that employ, use, and/or demonstrate how to use their own surgical instruments and/or implants.

The surgical interventions include surgical as well as minimally invasive surgical interventions.

The surgical interventions are implemented by the surgical instruments and/or implants provided.

The surgical interventions to be trained in include, for example, ablation of bone, insertion of medical implants, placement of medical screws, providing access, removal of tissue such as, for example, tumors, and providing surgical access to diseased regions.

The surgical interventions to be trained in also include minimally invasive methods such as, for example, medical endoscopy, minimally invasive spinal surgery (including spinal cord decompression and vertebral fusion after herniated discs), placement of implants and removal of tumors and/or bone adhesions.

An effective training for the user is advantageously possible by the system according to the invention. The training may be monitored in real time through the optoelectronic detection means. The individual anatomical design as well as the patient-specific data on the memory medium allow the user to focus on an individual and specific problem.

The training model advantageously includes replaceable components such as flushable bone-like material and pneumatized bones.

The typical surgical interventions to be practiced in the petrous bone region include mastoidectomy and cochleostomy training and the placement of implants such as, for example, middle and inner ear implants.

The artificial paranasal sinus patients enable above all the training in skull base surgery and functional endoscopic sinus surgery (FESS), in which, among other things, the opening of the sinuses and the removal of tumors (pituitary tumor) or polyps are among the frequent interventions to be trained in.

In spine surgery, training-intensive interventions are very common in spinal disc surgery for decompression or in the fixation of the vertebral body (e.g., after a fracture). The training model may also be used for learning minimally invasive surgical techniques and manual skills such as setting pedicle screws and intervertebral implants (cages), as well as the fusion of vertebrae.

The quality and learning success of the surgical interventions are validated through the use of the training model. This success monitoring immediately informs the user if any risk structures have been injured or if the operation has proceeded without disruption.

In embodiments of the invention, the components of the training model are designed to be reusable.

Depending on the type of training performed, all or some components of the training model are reusable. After training, the interchangeable practice region is removed from the anatomical reproduction of the body part and both components are cleaned separately. The anatomical reproduction of the body part is removed from the holder and the holder for the anatomical reproduction of the body part is cleaned as well. Furthermore, the surgical instruments used are cleaned.

In order to implement the invention, it is also expedient to combine the above-described designs of the invention, as well as the embodiments and features of the claims with each other in any configuration.

The invention will be explained in greater detail below with reference to some exemplary embodiments. The exemplary embodiments are intended to describe the invention without limiting it.

The invention may be applied in almost all fields of human and veterinary medicine. The invention will be explained in greater detail with reference to drawings. In which:

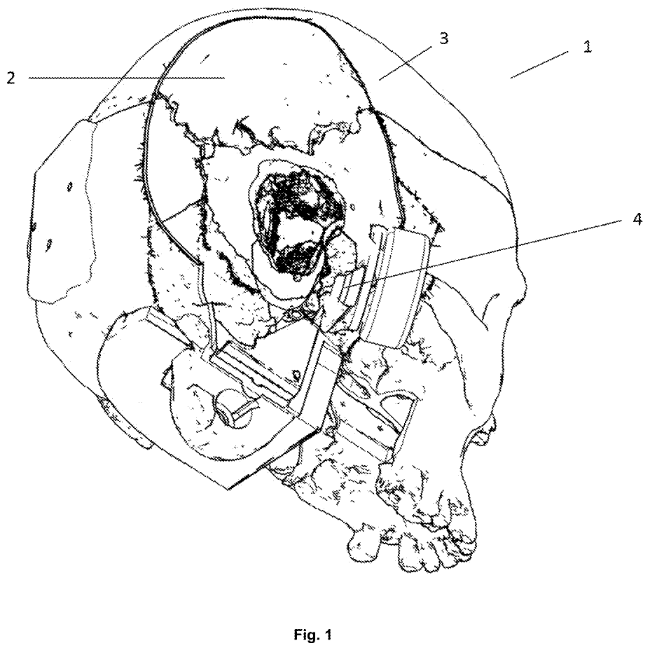

FIG. 1 shows an anatomical reproduction of a skull with an interchangeable petrous bone region and optoelectronic detection means,

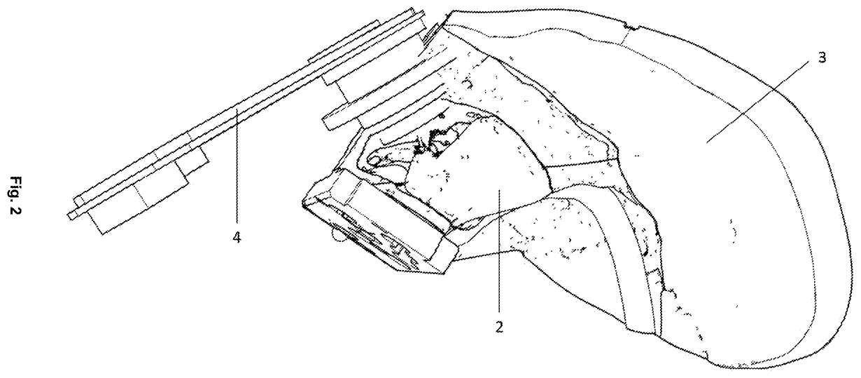

FIG. 2 shows a detailed view of the anatomical reproduction of a skull with an interchangeable petrous bone region and optoelectronic detection means,

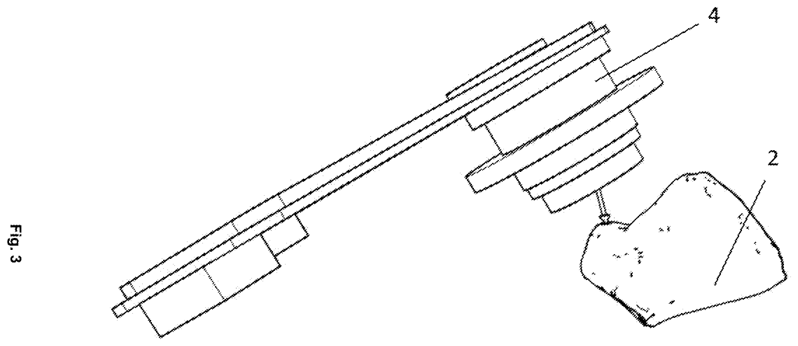

FIG. 3 shows a single view of the interchangeable petrous bone region with optoelectronic detection means,

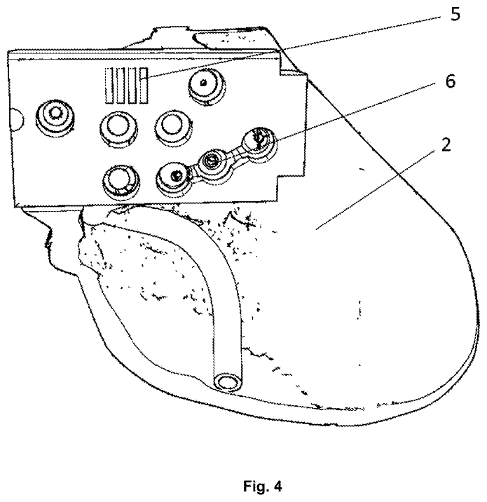

FIG. 4 shows an interchangeable petrous bone region with a memory medium and connections for the signal transmission means to the electronic control, measuring and evaluation unit,

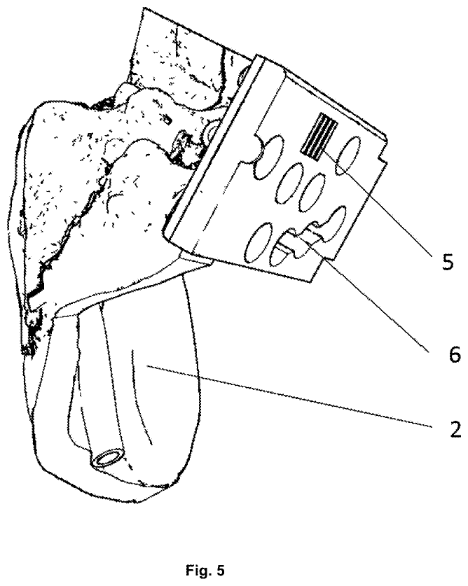

FIG. 5 shows the interchangeable petrous bone region from FIG. 4 in a side view,

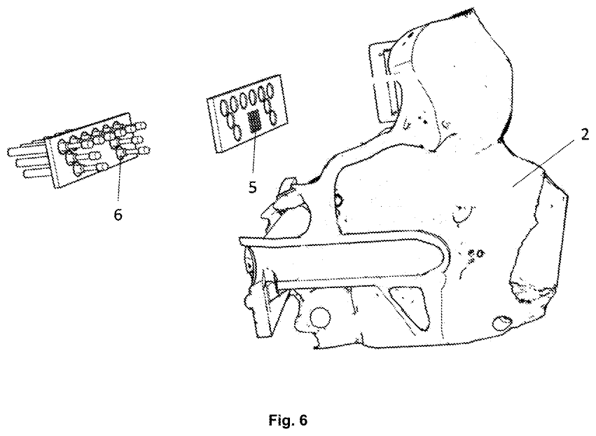

FIG. 6 shows an interchangeable nose region from the side in an exploded view with a memory medium and connections for the signal transmission means to the electronic control, measuring and evaluation unit,

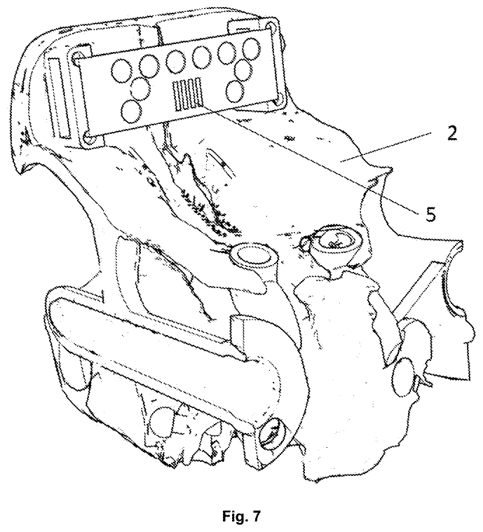

FIG. 7 shows the interchangeable nose region from FIG. 6 in a front view,

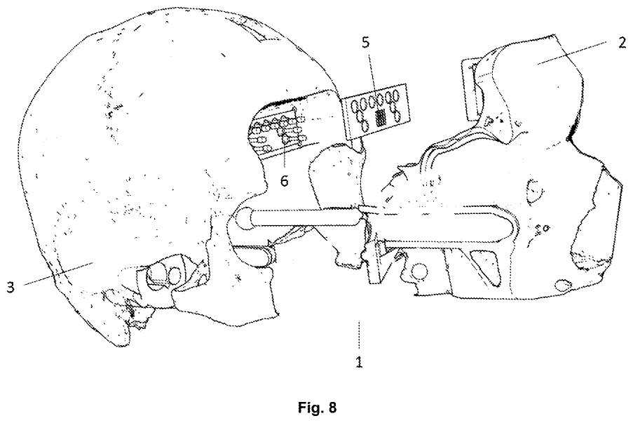

FIG. 8 shows an anatomical reproduction of a skull with interchangeable nose region and memory medium and connections for the signal transmission means to the electronic control, measuring and evaluation unit.

The first exemplary embodiment relates to the petrous bone region and thus to a limited area of the sphere of ENT physicians. The training model is used for implanting an electrode of a cochlear implant and comprises the anatomical reproduction of a human skull, in the recess of which an anatomically shaped and interchangeable petrous bone region may be inserted. The petrous bone region is precisely connected to the skull.

The petrous bone region in this case comprises a portion of the temporal bone (os temporale) and surrounds the inner ear (labyrinth). The petrous bone region also includes the pars tympanica (surrounding the middle ear) and the pars mastoidea of the temporal bone. The petrous bone region also includes the cochlea with a scala tympani and semicircular canals as well as (mobile) ossicles. The soft tissue is made of silicone such as, for example, the reproduction of the dura. The petrous bone region also comprises a reproduction of the eardrum and muscles and nerves such as, for example, the dacial nerve, chorda tympani and musculus stapedius. Blood vessels such as the jugular vein and the carotid artery are also reproduced.

The nervus facialis and the nervus tympanicus, among others, pass through the petrous bone region. A shallow pit for the ganglion trigeminal is located on the front surface (the inner surface in animals) of the petrous bone region. The petrous bone region has three important accesses: The internal acoustic meatus (inner ear opening) at which the facial nerve enters and the vestibulocochlear nerve exits, the stylomastoid foramen, at which the facial nerve exits and the canalis musculotubarius, to the channel of the auditory tube (eustachian tube) into the middle ear.

Furthermore, a column (petrotympanic fissure) between the petrous bone region and the tympanic part (tympanic) of the temporal bone serves as the outlet for the tympanic string (chorda tympani). In some mammals (for example, human, horse, cattle) the hyoid bone is attached via ligaments to the processus styloideus (styloid process) of the petrous bone.

The petrous bone region has a front side, which is freely accessible to the surgeon from outside, and a rear side. The rear side of the petrous bone region is at least partially in physical contact with the anatomical reproduction of the skull.

FIG. 1 shows a sketch overview of the system 1 according to the invention. The petrous bone region is shown as an interchangeable practice region 2. Said temporal bone region is inserted as a sensitively detected structure in the anatomical reproduction of a body part 3, in this case in a human skull, in an interlocking manner. An optoelectronic detection means 4, here a video digital camera, is connected via signal transmission means 7 (not shown) to the interchangeable practice region 2 and to an electronic control, measuring and evaluation unit 5 (not shown).

A (video) digital camera (not shown) is situated in the recess of the skull so that the surgical intervention is detected by a computer (not shown), and the positioning of the surgical instruments and/or implants (not shown) is monitored by the computer (not shown) on the rear side of the petrous bone region.

An SSD card serves as a memory medium (not shown). The SSD card is attached to the rear side of the interchangeable petrous bone.

All processes taking place are displayed on a monitor (not shown). In the process, the (video) digital camera sends a feedback signal to the computer (not shown).

FIG. 2 shows a detailed view of the system 1 of the anatomical reproduction of a skull 3 with an interchangeable region of the petrous bone of the inner ear 2 inserted in an interlocking manner therein as a sensitively detected (“glassy”) structure and an optoelectronic detection means 4.

FIG. 3 shows a single view of the petrous bone region of the inner ear 2 as a sensitively detected structure with an exposed optoelectronic detection means 4.

In a spine model as the second exemplary embodiment, a needle as a surgical instrument is stuck through a reproduction of the back portion (not shown). Practice hitting the nerve then takes place in the spine as an interchangeable practice region with the memory chip contained therein, which contains patient-specific data.

FIG. 4 shows the interchangeable practice region 2 with memory medium 5 and connections 6 for the signal transmission means to the electronic control, measuring and evaluation unit. The petrous bone region is shown as an interchangeable practice region 2. A memory medium 5, here an SSD card, is detachably situated in the rear side of the interchangeable practice region 2. Connections 6 for the signal transmission means to the electronic control, measuring and evaluation unit (not shown) are also attached to the rear side of the interchangeable practice region 2.

A (video) digital camera (not shown) is situated in the recess of the skull (not shown) so that the surgical intervention is detected by a computer, and the positioning of the surgical instruments and/or implants is monitored by the computer on the rear side of the petrous bone region. All processes taking place are displayed on a monitor (not shown). In the process, the (video) digital camera (not shown) sends a feedback signal to the computer (not shown).

FIG. 5 shows the interchangeable petrous bone region 2 from FIG. 4 from the side. Here too, the SSD card is again apparent as the memory medium 5, which is detachably situated in the rear side of the interchangeable petrous bone region 2. The connections 6 for the signal transmission means to the electronic control, measuring and evaluation unit (not shown) may also be seen on the rear side of the interchangeable petrous bone region 2.

In a nasal model of the nose region as a second exemplary embodiment, the ethmoidal cells are opened, for example, with various instruments, endoscopes and medical devices. The memory chip of the interchangeable practice region may include here, for example, the patient information such as age, medical history, patient anatomy and the behavior of the artificial patient in the event of a risk structure being injured. Furthermore, suggestions for the further course of the operation may be read out from the memory chip.

FIG. 6 shows in a second exemplary embodiment an interchangeable practice region 2, which represents the nose region, from the side. Also shown in the exploded view is the memory medium 5, which is designed as an SSD card, as well as connections 6 for the signal transmission means to the electronic control, measuring and evaluation unit (not shown), which are shown as pins.

FIG. 7 shows the interchangeable nose region 2 from FIG. 6 in a front view, now with a detachably inserted SSD card as the memory medium 5.

FIG. 8 shows a sketch overview of the system 1 according to the invention. The petrous bone region is shown as an interchangeable practice region 2. This may be inserted as a sensitively detected structure in the anatomical reproduction of a body part 3, in this case a human skull, in an interlocking manner. An SSD card as a memory medium 5 and connections for the signal transmission means (not shown) to the electronic control, measuring and evaluation unit (not shown) are delineated in an exploded view and are detachably inserted into the interchangeable nose region 2.

In a spine model as the third exemplary embodiment, a needle as an operative instrument is stuck through a reproduction of the back part (not shown). Practice hitting the nerve then takes place in the spine as an interchangeable practice region with the memory chip contained therein, which contains patient-specific data.

### REFERENCE NUMERALS

1 System for validating and training surgical interventions in human and veterinary medicine
2 Interchangeable practice region
3 Anatomical reproduction of a body part
4 Optoelectronic detection means
5 Memory medium
6 Connections for the signal transmission means to the electronic control, measuring and evaluation unit

1. A system for validating and training surgical interventions in human or veterinary medicine, which includes a training model anatomically modelled after a human or animal body, comprising
an anatomical reproduction of a body part of the human or animal body which has a recess,
an anatomical reproduction of an interchangeable practice region, which is designed to be inserted into the recess of the anatomical reproduction of the body part and
which has a front side, which is accessible from the outside, and
a rear side, which is connected at least partially in an interlocking manner to the recess of the anatomical reproduction of the body part, as well as
an optoelectronic detection means,
wherein the optoelectronic detection means is situated in the recess of the anatomical reproduction of the body part in such a way that it is designed to detect the surgical interventions and to monitor the positioning of surgical instruments and/or implants on the rear side of the interchangeable practice region.

2. The system according to claim 1, wherein the detection of the surgical interventions as well as the monitoring of the positioning of the surgical instruments and/or the implants on the rear side of the interchangeable practice region are implemented by an electronic control, measuring and evaluation unit.

3. The system according to claim 1, wherein the system comprises signal transmission means.

4. The system according to claim 1, wherein the optoelectronic detection means is a video digital camera having a CMOS sensor or a CCD sensor.

5. The system according to claim 1, wherein at least portions of the interchangeable practice region are transparent.

6. The system according to claim 1, wherein the interchangeable practice region comprises a memory medium, on which patient-specific data are stored and which is writable or rewritable with additional patient-specific data, wherein the memory medium is detachably arranged in the interchangeable practice region.

7. The system according to claim 1, wherein the optoelectronic detection means and/or the memory medium is/are connected by signal transmission means to the control, measuring and evaluation unit.

8. The system according to claim 1, wherein the interchangeable practice region is detachably connected to the anatomical reproduction of the body part.

9. An anatomical reproduction of an interchangeable practice region, wherein it is designed to be inserted in a holding device and
which has a front side, which is accessible from the outside, and
has a rear side, which is connected to the holding device in an interlocking manner,
wherein the interchangeable practice region comprises an optoelectronic detection means and a memory medium and at least portions of the interchangeable practice region are designed to be partially transparent.

10. The anatomical reproduction of an interchangeable practice region according to claim 9, wherein it comprises signal transmission means.

11. The anatomical reproduction of an interchangeable practice region according to claim 9, further comprising a memory medium.

12. A method for validating and training surgical interventions in human or veterinary medicine using a training model anatomically modelled after a human or animal body according to claim 2, wherein the optoelectronic detection means mounted in the interchangeable practice region of the training model sends a feedback signal to the control, measuring and evaluation unit.

13. The method according to claim 12, wherein the method for validating and training is monitored and evaluated in real time by the feedback signal of the optoelectronic detection means.

14. The method according to claim 12, wherein the interchangeable practice region comprises a memory medium, on which patient-specific data are stored and which is writable or rewritable with additional patient-specific data, wherein the memory medium is detachably arranged in the interchangeable practice region, and wherein the anatomical reproduction of the body part is connected to the interchangeable practice region and the memory medium contained in the interchangeable practice region is recognized by the electronic control, measuring and evaluation unit by signal transmission means.

15. The method according to claim 13, wherein the interchangeable practice region comprises a memory medium, on which patient-specific data are stored and which is writable or rewritable with additional patient-specific data, wherein the memory medium is detachably arranged in the interchangeable practice region, and wherein the anatomical reproduction of the body part is connected to the interchangeable practice region and the memory medium contained in the interchangeable practice region is recognized by the electronic control, measuring and evaluation unit by signal transmission means.

16. The system according to claim 2, wherein the system comprises signal transmission means.

17. The system according to claim 2, wherein the optoelectronic detection means is a video digital camera having a CMOS sensor or a CCD sensor.

18. The system according to claim 2, wherein at least portions of the interchangeable practice region are transparent.

19. The system according to claim 2, wherein the interchangeable practice region comprises a memory medium, on which patient-specific data are stored and which is writable or rewritable with additional patient-specific data, wherein the memory medium is detachably arranged in the interchangeable practice region.

20. The system according to claim 2, wherein:
the optoelectronic detection means and/or the memory medium is/are connected by signal transmission means to the control, measuring and evaluation unit; or
the interchangeable practice region is detachably connected to the anatomical reproduction of the body part.
